(19) United States
(12) Patent Application Publication
(10) Pub. No.: US 2024/0082046 A1
(43) Pub. Date: Mar. 14, 2024

(54) Ostomy Pouch

(71) Applicant: ConvaTec Limited, Flintshire (GB)

(72) Inventors: Clare Holden, Flintshire (GB); Alice Young, Flintshire (GB); Kevin Evans, Flintshire (GB)

(21) Appl. No.: 18/518,077

(22) Filed: Nov. 22, 2023

Related U.S. Application Data
(63) Continuation of application No. PCT/GB2022/051368, filed on May 27, 2022.

(30) Foreign Application Priority Data
Jun. 1, 2021 (GB) 2107773.0

(51) Int. Cl. A61F 5/445; A61F 5/44

(52) U.S. Cl. CPC A61F 5/445; A61F 5/4407

(57) ABSTRACT

An ostomy pouch, comprises: inner and outer walls of flexible sheet material defining a cavity for containing a stomal output; a deployable drain for draining stomal output from the cavity; and at least one sheet of comfort material. The drain moves between an extended configuration for draining the stomal output and a retracted configuration for storage of the drain. A first sheet of comfort material is attached to the outer wall, and a portion of the first sheet of comfort material forms a flap. The flap folds along a fold line, between a covering position and an access position; and the fold line is located between the deployable drain in the retracted position and an uppermost edge of the ostomy pouch.

### TECHNICAL FIELD OF THE INVENTION

The present invention relates to an ostomy pouch, and in particular to an ostomy pouch which is drainable.

### BACKGROUND TO THE INVENTION

An ostomy pouch may be used to collect and hold stomal output from a stoma formed in the body of an ostomate. Generally the stoma is a surgical opening in the torso of the ostomate's body, but may also refer to internal tissue, organs or portions thereof that are exposed by the opening. Ostomy pouches typically take the form of a pair of walls sealed together to form a cavity into which stomal output may be expelled from the ostomate through a formed stoma.

Given the nature and use of ostomy pouches, it is desirable for a pouch to be able to be worn by an ostomate as easily and discreetly as possible. It is also advantageous for the pouch to be worn for an extended period of time for increased convenience for the ostomate. For this reason, some ostomy pouches may be provided with a drain allowing the ostomate to intermittently drain stomal output from the cavity. Drains are typically moveable in some manner (e.g. by folding and/or rolling) between a stowed position where the pouch is closed, and a deployed position wherein the drain is open allowing the contents of the pouch to be drained therefrom—e.g. by opening a drain aperture in the pouch.

It may be desirable for a user for the contents of th ostomy pouch and or drain to be concealed during normal use (the ability to identify a drainable device being an undesirable characteristic, as the draining process is considered somewhat unpleasant).

Accordingly, some known solutions include an arrangement of some sort to hide the drain, for example in the Esteem®+Convex Invisiclose Drainable Pouch with Lock-it Pocket™ pouch by ConvaTec, arrangements have been provided which have an additional layer, or plurality of layers of comfort material arranged at the base of the pouch, forming a pocket, so as to allow for the drain to be folded up, then hidden when then the pocket is inverted. However, such pouches require additional manufacturing steps and suffer from increased complexity from a usability point of view, and in some instances may require the ostomate to use two hands to fully stow (and conceal) the drain. For example, the Lock-it Pocket™ is closed by placing the thumbs into the bottom corners of the Lock-it Pocket™ and turning it inside out, as if closing a money bag, to hold drain securely. Whilst this certainly improves discretion and security, the product still has a flat base, which takes its shape from the shape of the folded-up drain and is thus identifiable as drainable devices (which is considered an undesirable characteristic as the draining process is considered somewhat unpleasant).

The SALTs Confidence BE drainable pouches provide an alternative in which the base of an opaque comfort layer and adjacent opaque film layer is welded to the periphery of the two film layers that form the cavity, except at the base, thus forming a small flap, behind which th drain can be hidden when the drain is stowed/folded and introduced into the aperture formed between the flap and the cavity. However, because of the way this flap is formed, by welding with the film layers that form the cavity, there is a risk of compromising the integrity of the pouch. Specifically, the inventors have determined that because the fold line about which the flap is folded overlies the stowed drain, the user may apply excessive force to expose the drain, which in turn could result in the bonding of the covering layer being damaged. Damage to the pouch is undesirable as it may result in the pouch being prematurely discarded, the damage being perceived as interfering with the function of the pouch.

It is an aim of an embodiment or embodiments of the invention to overcome or at least partially mitigate one or more problems with the prior art.

### SUMMARY OF THE INVENTION

According to a broad aspect of the invention there is provided an ostomy pouch, comprising a deployable drain and a flap of comfort material configured to fold along a fold line between a covering position and an access position; wherein the fold line is located between the deployable drain in a retracted position and an uppermost edge of the ostomy pouch (i.e. located above the deployable drain when retracted).

The ostomy pouch may comprise inner and outer walls of flexible sheet material. The inner and outer walls may define a cavity for containing a stomal output. The ostomy pouch may comprise at least one sheet of comfort material. The deployable drain may be moveable between an extended configuration for draining the stomal output from the cavity and a retracted configuration for storage of the deployable drain. A first sheet of comfort material may be attached to the outer wall. At least a portion of the first sheet of comfort material may form a flap. The flap may be configured to fold along a fold line, between a covering position in which the flap covers the deployable drain when the deployable drain is in the retracted configuration, and an access position, in which the deployable drain, in the retracted configuration is uncovered by the flap and is able to be accessed for deployment.

According to an aspect of the invention there is provided an ostomy pouch, comprising: inner and outer walls of flexible sheet material defining a cavity for containing a stomal output; a deployable drain for draining stomal output from the cavity; and at least one sheet of comfort material; wherein the deployable drain is moveable between an extended configuration for draining the stomal output from the cavity and a retracted configuration for storage of the deployable drain; wherein a first sheet of comfort material is attached to the outer wall, at least a portion of the first sheet of comfort material forming a flap; the flap configured to fold along a fold line, between a covering position in which the flap covers the deployable drain when the deployable drain is in the retracted configuration, and an access position, in which the deployable drain, in the retracted configuration is uncovered by the flap and is able to be accessed for deployment; and wherein the fold line is located between the deployable drain in the retracted position and an uppermost edge of the ostomy pouch.

Advantageously, the location of the fold line between the deployable drain in the retracted position and the uppermost edge of the ostomy pouch (that is such that the fold line does not overlie the deployable drain) means that when the flap is folded up, the entire (retracted) drain can be accessed (unlike in the SALTS Confidence BE® product mentioned above). This allows for easy access to the deployable train, reducing the risk of damage to the bond of the comfort material. This in turn may reduce the risk of damaging the integrity of the pouch during draining. Since the risk of damage is reduced, the lifespan of individual pouches may increase, reducing the number of pouches required. This may produce an environmental benefit in reducing the amount of environmental waste produced.

Optional features set out below may apply to any aspect of the invention as appropriate.

The inner and outer walls may define a deployable drain for draining stomal output from the cavity. The drain may comprise an elongate extension of the inner and outer walls. The inner and outer walls may together define an elongate drain channel which extends from a drain inlet at the cavity and a drain aperture at a distal end of the drain channel through which stomal output from within the cavity can be drained from the pouch.

The inner and outer walls may be sealed (e.g. welded) about the periphery thereof to define the cavity and/or the drain. The seal/weld may comprise a single continuous edge seal. The single continuous edge seal may extend from a first edge (e.g. a left-hand edge) of a drain channel to a second edge (e.g. a right-hand edge) of a drain channel of the pouch. The single continuous edge seal may comprise a weld, which may optionally have a width of between 2 mm to 6 mm, or between 3 mm to 5 mm, or approximately 4 mm, for example. The single continuous edge seal may have a constant width around the periphery of the ostomy pouch.

The first sheet of comfort material may be joined to the outer wall at least about a part of the periphery. The joining may be by use of welding, adhesive or equivalent means. The weld attaching the first sheet of comfort material to the outer wall may be contiguous with the weld which defines the cavity. A single joining operation may be used to join the inner wall, the outer wall and the first sheet of comfort material together. For example, a single weld may be used to join the three layers. The first sheet of comfort material may be directly attached to the outer wall at the periphery thereof.

The fold line may be defined by a line extending between two points about the periphery where the attachment between the first sheet of comfort material and the outer wall terminates. The fold line may be defined by further points of attachment between the first sheet of comfort material and the outer wall. The fold line may be arranged at a distance of between 20 mm and 50 mm, or between 30 mm and 40 mm, for example about 35 mm, e.g. approximately 34 mm from a lower most edge of the pouch when the drain is in the retracted position.

The flap may be configured to fold around the fold line such that to move to an access position the end of the flap distal from the fold line is lifted away from the pouch and towards the uppermost edge of pouch. The flap may be configured to fold around the fold line such that to move to a covering position the end of the flap distal from the fold line is moved towards the lowermost edge of the pouch. The lowermost edge of the pouch may be defined by an interface between the drain and the cavity. The interface between the drain and the cavity may be defined by a fold line between the drain and the cavity.

The flap may be shaped such that the end distal from the fold line conforms to the shape of the periphery of the ostomy pouch. The flap may be shaped so as to correspond to one or more contours of the ostomy pouch. For example, in some embodiments the pouch may comprise one or more curved edges, and the flap may be similarly curved to correspond the contours of the curved edge(s) of the pouch. The distal end of the flap may be heated to give the appearance of welding (such that it matches a weld line around the periphery of the pouch.

The first sheet of comfort material may comprise a first part and a second part which may be joined to the outer wall so that the first part partially overlaps the second part, or the second part partially overlaps the first part in an overlap region. The first part and the second part may be separable from each other in the overlap region to form a window opening for viewing the cavity. The overlap region may be angled to extend horizontally when the pouch is in use. The first part and the second part of the first sheet of comfort material may be configured to slide over each other in the overlap region to accommodate expansion of the underlying outer wall. The first part and the second part may be joined to each other at a first end and at a second end of the overlap region. The first part and the second part may be welded to each other at the first end and at the second end of the overlap region, optionally as part of a peripheral weld of the pouch. External edges of the one or more parts of the first sheet of comfort material may be shaped and sized to correspond to the shape, form and contours of the outer wall.

The ostomy pouch may comprise a second sheet of comfort material. The second sheet of comfort material may be attached to the inner wall. The ostomy pouch may be provided with an opening between the second sheet of comfort material and the inner wall. The opening may be proximate to the deployable drain. The or each sheet of comfort material may be attached to the respective wall about a periphery thereof. An opening may be provided by a gap in the attachment in a region of the periphery.

Where present, the second sheet of comfort material may comprise a single part or multiple parts. The second sheet of comfort material may cover only a portion of the inner wall. However, preferably the second sheet of comfort material covers substantially the whole of the inner wall. An aperture may be provided in the inner comfort layer corresponding to the location of the inlet in the inner wall of the pouch. The inner comfort layer may be shaped and sized to correspond to the shape, form and contours of the inner wall. The inner comfort layer may be provided with a wafer aperture that corresponds to the location of the inlet of the inner wall to permit fluid connection of the inlet of the inner wall to an ostomy wafer.

The deployable drain may be moveable between stowed and deployed positions by rolling or folding the drain, for example, about one or more fold lines in the drain. The drain may be moved, in use, from the deployed position to the stowed position by folding and/or rolling the drain in a direction which is upwards and away from the ostomate.

In the retracted position the drain may extend between 20 mm and 50 mm from the lowermost edge of the pouch, or between 25 mm and 40 mm, for example about 30 mm from the lowermost edge of the pouch. In particular, that is the distance to the upper edge of the retracted drain (the top of the drain), e.g. as defined by the penultimate fold may extend between 20 mm and 50 mm from the lower most edge of the pouch, or between 25 mm and 40 mm, for example about 30 mm from the lowermost edge of the pouch.

The fold line may be arranged at least 0.5 mm; at least 1 mm; at least 2 mm; at least 3 mm; or at least 5 mm; at least 7.5 mm; at least 1 cm; or at least 2 cm above the upper edge of the retracted drain. The fold line may be arranged no more than 5 cm; no more than 3 cm; no more than 2 cm; or no more than 1 cm above the upper edge of the retracted drain, for example, preferably between 1 mm and 10 mm, more preferably about 4 mm. Such an arrangement of the fold line above the upper edge of the drain in the retracted position allows that the fold line to be arranged between the top of the drain and the upper most edge of the pouch.

The ostomy pouch may further comprise a fastener arrangement configured to retain the deployable drain in the retracted position. The fastener arrangement may comprise a first fastener located on an exterior surface (preferably the inner wall) of the deployable drain. The fastener arrangement may comprise a second fastener located on an exterior surface (in particular the outer wall) of the cavity. The second fastener may be provided on a tab. The tab may be secured to the outer wall of the cavity. The entire tab may be exposed when the flap is in the access position. The tab may comprise a fold line about which the tab is configured to fold between a fastened configuration and an unfastened configuration. The fold line about which the flap is configured to fold may be arranged between (a) the fold line about which the tab is configured to fold and (b) the uppermost edge of the ostomy pouch. This is beneficial as when the tab is pulled up it will not put excessive force on the fold between the flap and the cavity.

The fold line about which the tab is configured to fold may be at least 0.1 mm; at least 0.2 mm; at least 0.5 mm; at least 1 mm; or at least 2 mm below the fold line about which the flap is configured to fold. The fold line about which the tab is configured to fold may be no more than 10 mm; no more than 5 mm; no more than 2 mm; below the fold line about which the flap is configured to fold. For example the fold line about which the tab is configured to fold may preferably be between 2 mm and 7 mm, more preferably about 5 mm from the fold line about which the flap is configured to fold.

In embodiments, the fasteners may be hook-and-loop or hook-and-hook fasteners; for example first fastener may comprise a strip of hook fasteners and the second fastener may comprise a strip of loop fasteners together forming a hook and loop fastener arrangement. Equally, the second fastener may comprise a strip of loop fasteners and the first fastener may comprise a strip of hook fasteners. The first and second fasteners may each comprise a strip of hook fasteners forming a dual hook fastener arrangement. Alternative fasteners could include a resealable adhesive.

The drain may comprise one or more pursing strips. The pursing strips may comprise a rigid, bendable material. The pursing strip(s) may be associated with the inner or outer wall of the pouch. For example, the pursing strips may be adhesively or otherwise fixed to the inner or outer wall of the pouch. The pursing strip(s) may assist in separating the inner and outer walls of the pouch, in a portion thereof, to define a drain aperture in the pouch for draining stomal output from the cavity. For example, in use, the pursing strips may bend or arch under the application of a force by the ostomate/user. The bending or arching the strips may cause a corresponding movement of the inner and/or outer walls of the pouch to define an opening (i.e. a drain aperture) within the pouch.

In embodiments, the drain may comprise a pair of pursing strips. The pair of pursing strips may be provided on opposing sides of the drain. For example, the drain may comprise a first pursing strip on the inner wall and a second pursing strip on the outer wall. The pursing strips may overlie one another. Alternatively, the pursing strips may be offset.

In embodiments, the drain may be sub-divided into two or more segments. The segments may be defined by fold lines about which the drain may be folded when moving between deployed and stowed positions. For example, the drain segments may be sequentially folded to overlie one another in moving from the deployed position to the stowed position. The drain may comprise at least two segments, with a first pursing strip positioned on the inner wall of the pouch and defining, at least in part a first segment of the drain, with a second pursing strip positioned on the outer wall of the pouch and defining, at least in part a second segment of the drain. The drain may be sub-divided into three segments, with the second fastener provided on the inner wall of the pouch and defining, at least in part, a third segment of the drain.

The drain may be provided in a lower section of the pouch, e.g. depending from the bottom of the pouch when the pouch is configured to be used in a substantially vertical orientation.

The flexible sheet material of the inner and outer walls may comprise a single layer or a laminate of a plurality of layers. The flexible sheet material of the inner wall and/or the outer wall may comprise polyvinylidene chloride (PVDC) and/or ethylene-vinyl acetate (EVA). The inner wall and/or the outer wall may have a thickness of between 50 to 150 micrometres, or between 75 to 125 micrometres, or between 75 to 100 micrometres, for example.

The comfort material may be formed from a flexible sheet material. The material of the flexible sheet material may comprise one or more of polyester, nylon, viscose, polyurethane, polyethylene, polypropylene, polyvinylidene chloride (PVDC) and ethylene-vinyl acetate (EVA). The comfort layer may comprise a laminate of two or more layers. The comfort layer may comprise at least one fabric layer and at least one film layer. The at least one film layer may be laminated to the at least one fabric layer, and optionally may be laminated to the at least one fabric layer over an entire area of the at least one comfort layer. The at least one fabric layer may comprise a woven or a non-woven textile layer. The fabric layer may comprise polyester, nylon, viscose, polyethylene or polypropylene. The at least one film layer may comprise polyurethane, polyvinylidene chloride (PVDC) or ethylene-vinyl acetate (EVA). The comfort layer may have a thickness of between 50 to 1000 micrometres, or between 60 to 500 micrometres, or between 75 to 300 micrometres, or between 100 to 200 micrometres, for example.

The comfort material (of the first sheet and/or the second sheet) may be a fabric layer, for example a non-woven layer or a woven layer. The fabric layer may comprise a hot-melt adhesive (e.g. a web of EVA). The hot-melt adhesive may be arranged on the inside surface of the comfort layer. The hot-melt adhesive may penetrate the fabric layer. The web may comprise a mass of adhesive with voids therein, so as to reduce the amount of material required.

The hot-melt adhesive coating may be applied as a web. The web of hot melt adhesive may comprise a mass and a plurality of voids in the mass. The web may be a lattice, mesh or grid. The web may be a net or dots. The voids may be regularly spaced. The voids may be irregularly spaced. The voids may be regularly shaped. The web may have a regular and consistent distribution of adhesive. The voids may be irregularly shaped. The web may have an irregular and inconsistent distribution of adhesive. Each void may contain an absence (i.e. a substantial absence) of hot-melt adhesive applied to the comfort layer compared to the mass. The voids may make up at least 20%, at least 30%, at least 40%, at least 50%, at least 60%, or at least 70% of the web.

A web of this type can thus be much thinner and/or made of much less material than a full film, as well as avoiding the rustling associated with films.

The hot-melt adhesive may comprise one or more of a polyolefin, ethylene-vinyl acetate (EVA), polyurethane, polyvinylidene chloride (PVDC), silicon rubber, fluoropolymers, polycarbonate, styrene block co-polymer, polyester, polyamide, or polycaprolactone. In particular it may comprise EVA or co-polyester. In one embodiment the hot-melt adhesive is a polyolefin. In one embodiment the hot-melt adhesive is ethylene-vinyl acetate (EVA). In one embodiment the hot-melt adhesive is polyurethane. In one embodiment the hot-melt adhesive is polyvinylidene chloride (PVDC). In one embodiment the hot-melt adhesive is silicon rubber. In one embodiment the hot-melt adhesive is a fluoropolymer. In one embodiment the hot-melt adhesive is polycarbonate. In one embodiment the hot-melt adhesive is styrene block co-polymer. In one embodiment the hot-melt adhesive is polyester. In one embodiment the hot-melt adhesive is polyamide. In one embodiment the hot-melt adhesive is polycaprolactone. The hot melt-adhesive may have an area density of at least 5, 10, 15, 16 or 20 g/m², and/or no more than 50, 40, 35, 30 or 25 g/m² preferably 10-50 g/m², 16-35 g/m², 20-30 g/m², or 23-27 g/m², for example 25 g/m².

The hot-melt adhesive may have a maximum thickness, or an average (mean) thickness of less than 0.05 mm, less than 0.04 mm, less than 0.03 mm, less than 0.02 mm, or less than 0.01 mm, for example less than 0.005 mm. Providing the hot-melt adhesive as a thin layer/web reduces the amount required, with corresponding benefits in terms of cost, weight of starting material required, etc.

The inside surface of the sheet of comfort material may be divided into one or more regions. The hot-melt adhesive may be applied to the region or one or more regions. Where there are two or more regions, specific properties of the web may be selected for each region. One or more of the regions may not have hot-melt adhesive applied to them.

The first sheet of comfort material may comprise an outer surface and an inner surface wherein at least part of the inner surface (preferably at least including the flap) is coated with a web of hot-melt adhesive.

The inner wall of the pouch may comprise an inlet for receiving the stomal output into the cavity.

The pouch may comprise either an ostomy wafer that is located within or otherwise associated with the inlet of the inner wall, or a releasable coupling that is located within or is otherwise associated with the inlet of the inner wall. In embodiments, the releasable coupling may be configured for coupling with a body fitment component comprising an ostomy wafer. Where present, the ostomy wafer may extend through an aperture of the inner wall and/or inner comfort layer. The ostomy wafer may be provided with a releasable liner which may be removed by a user prior to securing the pouch to the ostomate, in use.

In embodiments, the cavity may comprise one or more sections, for example and upper and a lower section. The cavity may comprise a waisted section located between the upper and lower sections. The waisted section may comprise a minimum width which is less than the maximum width of the upper and/or lower sections.

In embodiments, the cavity may comprise the upper section, the lower section and the waisted section located between the upper section and the lower section.

The upper and/or lower sections may be generally rounded in shape. For example, the upper section may comprise a continuously curved edge that extends from a first edge (e.g. a left-hand edge) of the waisted section to a second edge (e.g. a right-hand edge) of the waisted section. The lower section may comprise a continuously curved edge that extends from a first edge (e.g. a left-hand edge) of the waisted section to a second edge (e.g. a right-hand edge) of the waisted section.

The continuously curved edge of the upper and/or lower sections may be convexly curved. The continuously curved edge of the upper and/or lower sections may be absent any points of inflection or abrupt changes in contour.

A junction between the upper and/or lower sections and the waisted section may be demarcated by a single point of inflection between a left-hand edge of the upper or lower section and a left-hand edge of the waisted section, and by a single point of inflection between a right-hand edge of the upper or lower section and a right-hand edge of the waisted section.

According to another broad aspect of the invention there is provided a method of draining an ostomy pouch; the method comprising folding up a flap of comfort material, along a fold line located between a deployable drain in a retracted configuration and an uppermost edge of the ostomy pouch, into an access position to expose the deployable drain.

According to an aspect of the invention there is provided a method of draining an ostomy pouch; the method comprising folding up a flap of comfort material, along a fold line located between a deployable drain in a retracted configuration and an uppermost edge of the ostomy pouch, into an access position to expose the deployable drain; deploying the deployable drain into an extended configuration; draining the stomal output from the cavity; stowing the deployable drain into the retracted configuration; and folding the flap down, along the fold line, into a covering position to cover the deployable drain. The method may further comprise the step of releasing a fastener prior to deploying the deployable drain into the extended position. The method may further comprise the step of securing the deployable drain in the retracted configuration with a fastener. The method may further comprise the step of releasing a fastener prior deploying the deployable drain.

According to an aspect of the invention there is provided a method of collecting stomal output using an ostomy pouch according to any aspect described herein. The method may comprise attaching the ostomy pouch about a stoma of an ostomate. The ostomy pouch may be attached about the stoma through use of an ostomy wafer of the pouch where the pouch comprises a one-piece ostomy pouch. Alternatively, the method may comprise attaching an ostomy wafer of a body fitment component of a two-piece ostomy pouch about the stoma; and attaching a pouch to the body fitment component. The pouch may be attached to the body fitment component before or after the ostomy wafer has been attached about the stoma. Where the ostomy pouch comprises an open or drainable pouch, the method may comprise draining stomal output from the ostomy pouch. The ostomy pouch may be drained according to the method of the preceding aspect of the invention.

According to another broad aspect of the invention there is provided a method of forming an ostomy pouch, the method comprising; attaching a first sheet of comfort material to an outer wall of an ostomy pouch, leaving a flap arranged to cover a deployable drain when in the retracted position, the flap configured to fold along a fold line; wherein the fold line is located between the deployable drain in the retracted configuration and an uppermost edge of the ostomy pouch.

According to an aspect of the invention there is provided a method of forming an ostomy pouch, the method comprising; providing an inner wall and an outer wall which define a cavity for containing stomal output, and a deployable drain for draining stomal output from the cavity; wherein the deployable drain is moveable between an extended configuration for draining the stomal output from the cavity and a retracted configuration for storage of the deployable drain; attaching a first sheet of comfort material to the outer wall, leaving a flap arranged to cover the deployable drain when in the retracted position, the flap configured to fold along a fold line; wherein the fold line is located between the deployable drain in the retracted configuration and an uppermost edge of the ostomy pouch.

In any of the methods, the pouch may be the pouch of any aspect of the invention outlined above and may comprise any of the optional features disclosed above, or combinations thereof.

### DETAILED DESCRIPTION OF THE INVENTION

In order that the invention may be more clearly understood one or more embodiments thereof will now be described, by way of example only, with reference to the accompanying drawings, of which:

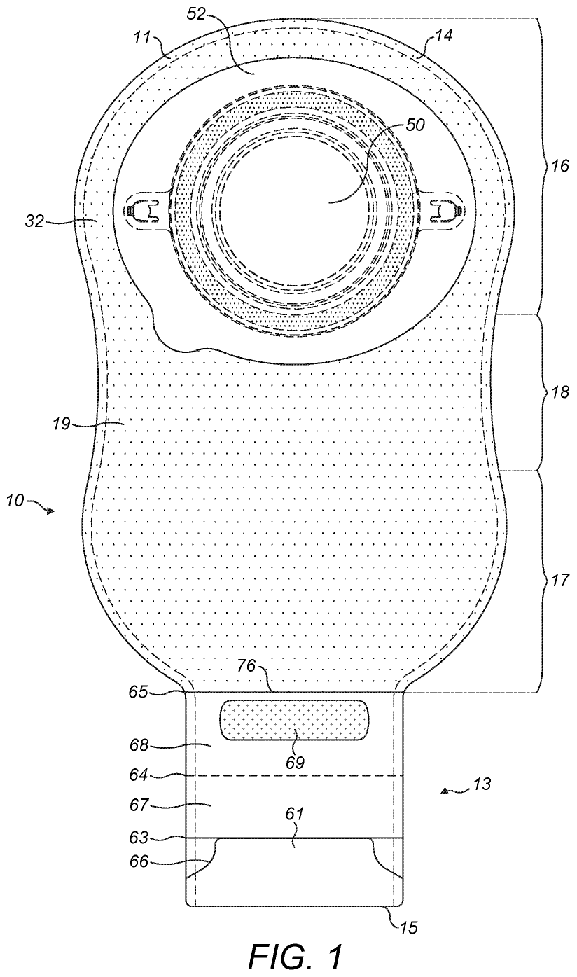

FIG. 1 is a perspective view of the body-side of an embodiment of an ostomy pouch in accordance with the invention;

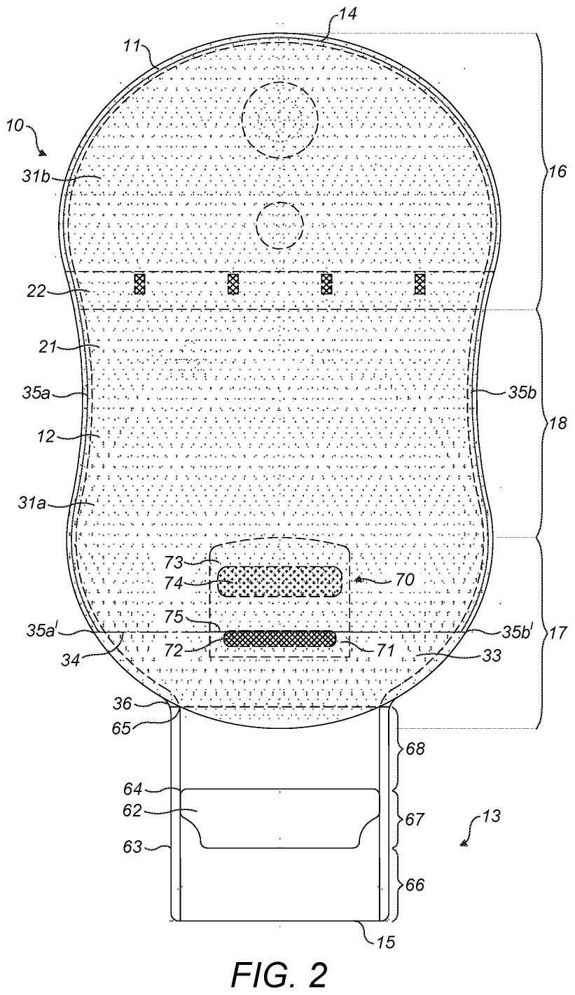

FIG. 2 is a perspective view of the clothing-side of the ostomy pouch of FIG. 1;

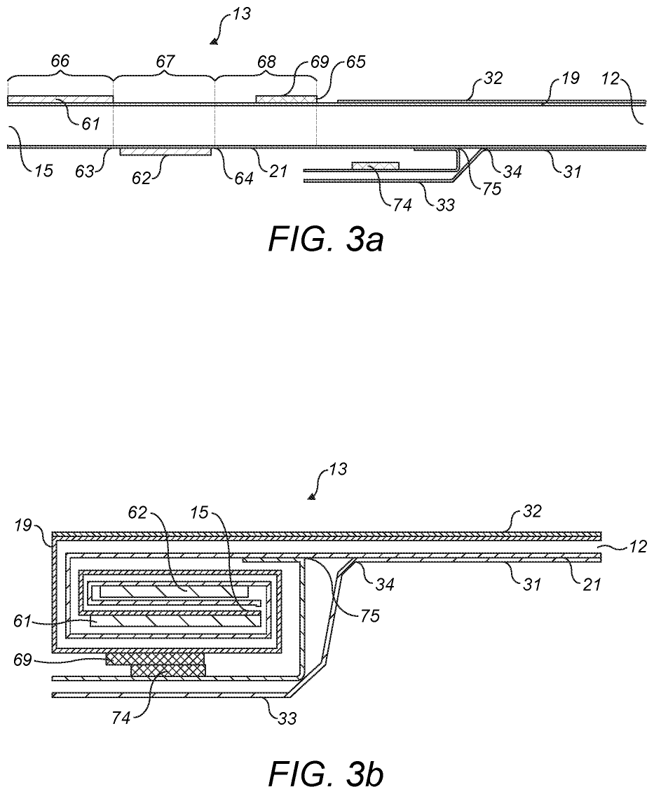

FIG. 3a is a cross-sectional schematic view of the drain of the ostomy pouch of FIG. 1 in a deployed position; and

FIG. 3b is a cross-sectional schematic view of the drain of the ostomy pouch of FIG. 1 in a retracted position.

When used herein and throughout the specification, the term “stomal output” refers to any gases, liquids or solids produced by an ostomate that may be secreted from a stoma or that exit a stoma of the ostomate. The stomal output may comprise gaseous, fluid, liquid and/or solid stomal output.

The term “stoma” refers to an opening in the body. Generally, the stoma is a surgical opening in the torso of the body. In some instances, the term “stoma” also refers to internal tissue, organs or portions thereof that are exposed by the opening. By way of non-limiting example, internal tissue may be selected from colon, ileum, small intestine, large intestine, jejunum, and duodenum, and combinations thereof. The internal tissue may be an end or a loop of a small or large intestine.

The term “ostomate” refers to a subject that may have use of the ostomy pouch described herein. While ostomate usually refers to a subject with a surgical opening, as used herein, “ostomate” may refer to a subject who has a stoma, regardless of whether the stoma was created by surgery or other means.

The term “user” may refer to an ostomate, or to another person assisting the ostomate, for example, with emptying of the stomal output from the cavity.

Ostomy pouches disclosed herein may, for example, be used for managing a stoma created by an esophagostomy, a gastrostomy, a cholecystectomy, a choledochostomy, a cecostomy, a colostomy, a duodenostomy, an ileostomy, a jejunostomy, an appendicostomy, a tracheostomy, a urostomy, a nephrostomy, a ureterostomy, or a vesicostomy. The ostomy pouches disclosed herein may be used with additional devices including, but not limited to, a shunt, a catheter, a plug or a faecal management system.

In this specification locations and orientations of features may be described with reference to the ostomy pouch being “in use”, “orientated as it would be in use” or similar. Such terms refer to the intended orientation of the ostomy pouch when it is adhered or otherwise secured to a body of an ostomate, e.g. with the ostomate in a standing position, irrespective of whether the ostomy pouch is currently performing such a use or the actual position of the ostomate. The terms “upper” and “lower” and related terms refer to the relative position of a part or portion of the ostomy pouch when orientated as it may be in use. For example, a section of the ostomy pouch may be referred to as an “upper” section of the ostomy pouch, and reference may be made to the uppermost edge of the pouch. In such an example, said section/edge will be intended to be the uppermost section (in the vertical direction) of the ostomy pouch when attached to the body of a standing ostomate. However, the reader skilled in the art will appreciate that before attachment to the ostomate said section may not always be the uppermost section and in addition when attached the section may not always be the uppermost section if the ostomate adopts a non-standing position, for example lying down.

The terms “left-hand” and “right-hand” and related terms may refer to the ostomy pouch when viewed from the rear (for example, as shown in FIG. 1). Thus, as an illustrative example, a “left-hand” edge of the ostomy appliance will be towards a left-hand side of the ostomate in the situation where the ostomy pouch is attached to the front torso of the ostomate.

The terms “concave” and “convex” and related terms refer to shaping of features of the ostomy pouch when viewed from an exterior of the ostomy pouch. Thus, as an illustrative example, an ostomy wafer of circular shape would be considered to have a convexly shaped peripheral edge.

The terms “inner” and “outer” refer to the relative position of a part or portion of the ostomy pouch with reference to the body of an ostomate when the ostomy pouch is attached (e.g. adhesively or otherwise) to the body of the ostomate. “Inner” refers to a position relatively closer to the body of the ostomate than a comparative position that is “outer”. “Outer” refers to a position relatively further away from the body of the ostomate than a comparative position that is “inner”. Inner is synonymous with “body-side” in the art, whilst outer is synonymous with “clothing-side”.

Ostomy pouches are commonly attached to the body of an ostomate by means of an ostomy wafer which includes an adhesive layer or layers. The ostomy wafer typically has an opening for the stoma sometimes referred to as a starter hole which may be cut to a required size by a user before attachment. The ostomy wafer typically comprises an adhesive layer on a body-facing side for adhering the ostomy wafer to the body of the ostomate. Typically, a release liner covers a body-facing side of the ostomy wafer that is removed by the user prior to fitting to the skin. In this specification, the term “ostomy wafer” may be used interchangeably with the terms “adapter,” “wafer,” “baseplate”, or “layered adhesive wafer.” The “ostomy wafer”, “adapter,” “wafer,” “baseplate”, or “layered adhesive wafer” may form a skin barrier between the ostomate and the pouch. In this specification, the term “ostomy wafer” includes ostomy wafers for a “two-piece appliance” and for a “one-piece appliance”.

A “two-piece pouch” refers to an ostomy pouch where the ostomy wafer forms part of a separate body fitment component that is attached by a releasable coupling to a pouch. A two-piece pouch permits the body fitment component to be separated from the pouch without damage, so that at least one of the parts continues to be functionally usable. For example, the body fitment component may remain in place on the body of the ostomate. In contrast, a “one-piece pouch” refers to an ostomy pouch where the ostomy wafer is permanently attached to the appliance, to the extent that the ostomy wafer cannot easily be separated without risk of damaging the appliance. A one-piece pouch is intended to be used as an integral unit.

Ostomy pouches may commonly be configured as “closed” pouches or “open” pouches. In this specification a “closed pouch” refers to an ostomy pouch where it is not intended that stomal output is drained from the cavity. Thus, a closed pouch may typically be configured as a one-use, disposable and non-reusable pouch. In this specification an “open pouch” refers to an ostomy pouch where it is possible for the stomal output to be drained from the cavity and the pouch reused. Thus, an open pouch may be configured as a reusable pouch, such that it can be reused and emptied multiple times whilst attached to the body, although this is not essential. In an open pouch the stomal output may be drained intermittently as instigated by an action of the ostomate or may be drained intermittently or continuously due to the cavity being fluidly connected to a drain, for example a night drain line.

The use of a closed pouch or an open pouch may be, in part, due to user preference, but equally either a closed or open pouch may be more suited depending on the particular ostomate's needs, and depending on the position of the stoma for the ostomate. For example, for stomas formed via ileostomy the stomal output may tend to be looser and be easily drainable which may lead to an open pouch being suitable. For stomas formed by colostomy, the stomal output may tend to be more solid and may not be readily drained by a user. In such instances, a closed pouch may be more suited.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

FIGS. 1 to 3 illustrate an exemplary ostomy pouch 10, having an inner wall 19 and an outer wall 21 sealed about at least part of the periphery 11 thereof to define a cavity 12 for containing a stomal output and a deployable drain 13. The pouch 10 is therefore “open”, with the seal 14 being provided as a single, continuous seal about the perimeter 11 of the pouch 10, except at a drain aperture 15, described in detail hereinbelow. In this embodiment the cavity includes a first, upper section 16 and a second lower section 17, which are generally rounded in shape with convex curved edges. The upper section 16 and lower section 17 are separated by a waist section 18 which is narrower in width than the upper section 16 and lower section 17. The waist section 18 has concave curved edges. The deployable drain 13 is provided extending from the lower section 17 of the pouch.

Those skilled in the art will readily appreciate that the shape of the ostomy pouch is not restricted to that described above, indeed the teachings disclosed here within could equally apply other pouch shapes, such as pouches with no waist section, or a plurality of waist sections.

The inner wall 19 and outer wall 21 of this embodiment are formed of a flexible plastic sheet material. The pouch 10 also includes a first sheet of comfort material forming an outer comfort layer 31 and a in this embodiment also includes second sheet of comfort material forming an inner comfort layer 32. The inner comfort layer 32 overlaying the inner wall 19 and the outer comfort layer 31 overlaying the outer wall 21. In this embodiment the comfort layers 31, 32 are formed of a woven fabric material and define the outer surfaces of the pouch 10. The surfaces of the inner and outer comfort layers 31, 32 facing the inner and outer walls 19, 21 respectively, are coated in a web of hot-melt adhesive (in this instance, EVA) so as to allow the comfort layers to be welded to the pouch without the need for intervening layers. However, it will be appreciated that the comfort layer could be formed of various other materials, could be provided as a plurality of layers and so forth.

In this embodiment the outer comfort layer is provided in two parts, a first part 31a covering the lower section 17, the waist section 18 and partially covering the upper section 16; and a second part 31b partially covering the upper section 16. The first and second parts partially overlap in an overlap region. The second part 31b is joined to the outer wall 21 about the periphery 11 thereof. The first part 31a is joined to the outer wall 21 along a part of the periphery thereof, the first part 31a is joined along a right periphery 35a and a left periphery 35b the joins terminating at lower weld ends 35a′ and 35b′. The lower edge of the first part 31a is not joined to the outer wall and instead forms a flap 33. The flap 33 folds along a flap fold line 34, the fold line extends between the lower weld ends 35a′, 35b′ of the right and left peripheral joins of the first part 31a. The flap 33 is shaped such that when in a covering position, wherein it covers the deployable drain (further elaborated on hereinbelow) in a retracted position, the edge 36 distal from the flap fold line 34 conforms with the shape of the pouch when the drain is in the retracted position.

The first and second parts 31a, 31b of the outer comfort layer are, in use, separable from each other in the overlap region 22 to form a window opening for viewing the cavity. Whilst in the illustrated embodiment, the second part 31b overlays the first part 31a in the overlap region, one skilled in the art will recognise that such an overlap could be formed in the opposite manner, that is the first part 31a overlapping the second part 31b in the overlap region 22, and of course in other embodiments, the outer comfort layer may be formed in one-part without an overlap.

The inner wall 19 comprises an opening 50 therein defining a stomal inlet 51 in the pouch 10 for receiving stomal output into the cavity. The stomal inlet 51 is arranged in the upper section 16 of the pouch. In this embodiment the inner comfort layer 32 is provided with an opening 35 arranged concentrically with the stomal inlet, thereby allowing an ostomy wafer 52 to be applied to the stoma in use. In this embodiment, the ostomy wafer 53 is permanently attached to the pouch, i.e. it is a one-piece pouch, but of course it could be separable (i.e. a two-piece pouch).

In this embodiment the weld between the inner wall and the inner comfort layer does not extend around the entire periphery thereof, a section 76 is proximate to the drain 13 remains unwelded such that a gap is formed between the inner wall and inner comfort layer.

As stated above, the inner and outer walls 19, 21 further define a deployable drain 13. The drain 13 is integral with the inner and outer walls and in this embodiment is elongate, extending from the lower edge of the lower section 17, with the weld about the periphery 11 extending along each edge of the drain to a drain aperture 15 where the pouch remains unsealed, i.e. a region of the periphery 11 where the inner and outer walls are not joined together. In use the drain aperture 15 may be used to remove stomal output from the cavity.

In this embodiment the drain 13 is separated into three equal sections 66, 67, 68 along its length by first and second fold lines 63, 64 extending laterally across the drain 13, a third fold line 65 extends laterally across the drain 13 at the interface of the drain and the cavity defining the lowermost edge of the pouch (when the drain is retracted). Arranged on a portion of the inner wall 19 defining the section 66 proximate to the drain aperture 15, this embodiment includes a first pursing strip 61. A second pursing strip 62 is arranged on a portion of the outer wall 21 defining the middle section 67 of the drain 13 of this embodiment. Arranged on a portion of the inner wall 19 defining the section 68 proximate to the cavity is a first fastening means 69, in this illustrative embodiment the fastening means is a strip of self-gripping fastener hook tape. Those skilled in the art will appreciate that other means of fastening are also suitable, that pursing strips are a desirable, but optional feature, and that other arrangements are within the ambit of the invention.

Arranged on the lower portion 17 of the outer wall 21 of this exemplary pouch is the second fastener 70. The second fastener 70 is arranged at the middle of and below the fold line extending between lower weld ends 35a′ and 35b′. The second fastener 70 of this particular embodiment is formed of a foldable foam tab, the tab folding around a tab fold line 75, the tab fold line defining two sections of the second fastener 70, an adhesive section 71 and a fastening section 73. One side of the adhesive section 71 is coated in an adhesive 72 which adheres the foam tab to the exterior surface of the outer wall 21. The fastening section 73 is provided with a second fastening means 74 (in this illustrative embodiment a strip of self-gripping fastener hook tape), arranged on the opposite surface of the tab to the adhesive 72 on the adhesive section. In this embodiment the foldable foam tab is arranged such that the fold line 75 thereof, is arranged parallel to, but below the fold line 34 of the flap 33 (that is the fold line of the foldable foam tab is arranged closer to the lower edge of the pouch).

In use the drain 13 is moveable between an extended configuration (as shown in FIG. 3a) and a retracted configuration (as shown in FIG. 3b). In this embodiment the drain 13 is moved from the extended configuration, to the retracted configuration in the following manner: First the section 66 of the drain proximate to the drain aperture 15 away from the body if folded about the first fold line 63 such that the sections of the outer wall 21 forming the middle section 67 of the drain and the section 66 proximate to the drain aperture 15 are in contact. Next the drain is folded away from the body about the second fold line 64 such that the section of the inner wall 19 forming the section 66 proximate to the drain aperture 15 and the section of the outer wall 21 forming the section 68 proximate to the cavity are in contact. A third fold is performed about the third fold line 65, again folding the drain 13 away from the body, such that section 62 of the inner wall 19 forming the middle section 67 is in contact with the outer wall 21 of the cavity. In the retracted configuration the top of the retracted drain, as formed by the second fold line 64 is arranged below the line extending between lower weld ends 35a′ and 35b′. One skilled in the art will appreciate that the drain could be provided with a greater number of, or fewer, sections to which a corresponding increase or decrease in the number of folds could be applied. Further it will be appreciated that as an alternative to folding the drain may instead be retracted in an alternative way, such as rolling.

When the drain 13 is in the retracted configuration, the repeated folds about the three fold lines 63, 64, 65 effectively seal the drain 13, such that no stomal output can escape the cavity. The drain can be held in the retracted configuration by folding the second fastener 70 about the fold line 72 such that the first and second fastening means 69, 74 come into contact. Thereafter the flap 33 can be folded down about the flap fold line 34 to cover the drain 13 in the retracted configuration.

Conversely, to allow draining of the pouch 10, the drain 13 can be moved from the retracted configuration to the deployed configuration. First the flap is folded back about the flap fold line 34 to fully expose the retracted drain 13 and fastener tab 70 (i.e. the flap fold line does not overlie the retracted drain or fastener tab). The fastener tab 70 can then be detached from the drain, and the folding action is undertaken in the reverse order, moving the drain from the retracted configuration to the extended configuration (i.e. unfolding about the third fold line 65, then the second fold line 64 and finally the first fold line 63) the drain 12 is no longer sealed and the stomal output can be drained from the cavity via the drain aperture 15.

Except in Examples, or where otherwise explicitly indicated, all numerical quantities in this description specifying amounts of materials, device dimension, and the like, are to be understood as modified by the word “about.”

The one or more embodiments are described above by way of example only. Many variations are possible without departing from the scope of protection afforded by the appended claims.

1. An ostomy pouch, comprising:
inner and outer walls of flexible sheet material defining a cavity for containing a stomal output;
a deployable drain for draining stomal output from the cavity; and
at least one sheet of comfort material;
wherein the deployable drain is moveable between an extended configuration for draining the stomal output from the cavity and a retracted configuration for storage of the deployable drain;
wherein a first sheet of comfort material is attached to the outer wall, at least a portion of the first sheet of comfort material forming a flap; the flap configured to fold along a fold line, between a covering position in which the flap covers the deployable drain when the deployable drain is in the retracted configuration, and an access position, in which the deployable drain, in the retracted configuration is uncovered by the flap and is able to be accessed for deployment;
wherein the fold line is located between the deployable drain in the retracted position and an uppermost edge of the ostomy pouch.

2. An ostomy pouch according to claim 1 wherein the first sheet of comfort material is attached to the outer wall at least about at least part of the periphery thereof.

3. An ostomy pouch according to claim 2 wherein the fold line is defined by a line extending between two points about the periphery where the attachment between the first sheet of comfort material and the outer wall terminates.

4. An ostomy pouch according to any preceding claim wherein the flap is shaped such that an edge distal from the fold line conforms to the shape of the periphery of the ostomy pouch.

5. An ostomy pouch according to any preceding claim wherein the comfort material forming the flap is coated with a hot melt adhesive.

6. An ostomy pouch according to any preceding claim further comprising a fastener arrangement configured to retain the deployable drain in the retracted position, the fastener arrangement comprising a first fastener located on an exterior surface of the deployable drain and a second fastener located on an exterior surface of the cavity.

7. An ostomy pouch according to claim 6 wherein the second fastener comprises a tab, which is secured to the outer wall of the cavity.

8. An ostomy pouch according to claim 7 wherein the tab comprises a tab fold line about which the tab is configured to fold between a fastened configuration and an unfastened configuration.

9. An ostomy pouch according to claim 8 wherein a foldable portion of the tab is exposed when the flap is in the access position.

10. An ostomy pouch according to claim 9 wherein the tab fold line is arranged between the fold line of the flap and a lower edge of the pouch.

11. An ostomy pouch according to claims 8 to 10 wherein the fold line of the flap is arranged between the tab fold line and the uppermost edge of the pouch.

12. An ostomy pouch according to any of claims 8 to 11 wherein the tab fold line is arranged at least 0.5 mm below the fold line of the flap.

13. An ostomy pouch according to any of claims 8 to 12 wherein the tab fold line is arranged at least 1 mm below the fold line of the flap.

14. An ostomy pouch according to any of claims 8 to 13 wherein the tab fold line is arranged no more than 10 mm below the fold line of the flap.

15. An ostomy pouch according to any of claims 8 to 14 wherein the tab fold line is arranged no more than 5 mm below the fold line of the flap.

16. An ostomy pouch according to any preceding claim wherein the fold line of the flap is arranged at a distance of between 20 mm and 50 mm from a lower edge of the pouch when the drain is in the retracted position.

17. An ostomy pouch according to claim 16 wherein the fold line of the flap is arranged at a distance of between 30 mm and 40 mm from a lower edge of the pouch when the drain is in the retracted position.

18. An ostomy pouch according to any preceding claim wherein the fold line of the flap is arranged at a distance of at least 1 mm above the upper edge of the retracted drain.

19. An ostomy pouch according to any preceding claim wherein the flap is configured to fold around the fold line such that to move to the access position the end of the flap distal from the fold line is lifted away from the pouch and towards the uppermost edge of the pouch.

20. An ostomy pouch according to any preceding claim wherein the pouch further comprises a window opening for viewing the cavity.

21. An ostomy pouch according to claim 20 wherein the window opening is arranged on the outer wall, the first sheet of comfort material comprising a first and second part, wherein the first part partially overlaps the second part, or the second part overlaps the first part in an overlap region to form the window.

22. A method of forming an ostomy pouch, the method comprising:
providing an inner wall and an outer wall which define a cavity for containing stomal output, and a deployable drain for draining stomal output from the cavity; wherein the deployable drain is moveable between an extended configuration for draining the stomal output from the cavity and a retracted configuration for storage of the deployable drain;
attaching a first sheet of woven comfort material to the outer wall, leaving a flap arranged to cover the deployable drain when in the retracted position, the flap configured to fold along a fold line;
wherein the fold line is located between the deployable drain in the retracted configuration and an uppermost edge of the ostomy pouch.
